(19) United States
(12) Patent Application Publication
(10) Pub. No.: US 2023/0162827 A1
(43) Pub. Date: May 25, 2023

(54) METHOD AND SYSTEM FOR PREDICTING NEUROLOGICAL TREATMENT

(71) Applicant: Inteneural Networks Inc., Chicago, IL (US)

(72) Inventors: Marek KRAFT, Poznan (PL); Paul LEWICKI, Tulsa, OK (US); Kris B, SIEMIONOW, Chicago, IL (US); Dominik PIECZYNSKI, Tulce (PL); Michal MIKOLAJCZAK, Poznan (PL); Mikolaj Andrzej PAWLAK, Poznan (PL); Michal KLIMONT, Poznan (PL)

(21) Appl. No.: 18/094,436

(22) Filed: Jan. 9, 2023

Related U.S. Application Data
(63) Continuation-in-part of application No. 16/885,315, filed on May 28, 2020, now abandoned.

(30) Foreign Application Priority Data
Jun. 1, 2019 (EP) 19177790.3

(51) Int. Cl. G16H 20/00; G16H 10/60

(52) U.S. Cl. CPC G16H 20/00; G16H 10/60; G16H 30/20

(57) ABSTRACT

A computer-implemented method for predicting neurological treatment for a patient. The method includes analyzing a pre-stored brain image of the patient by means of a Convolutional Neural Network to determine brain image analysis result including at least one of: a presence of a tumor or lesion, brain age, brain health, gyrification coefficient; receiving additional data, including at least one of: voice recognition index, additional symptom checks, blood work results, genetic sequencing results; and combining the brain image analysis result with the additional data to determine a score related to a probability that the patient may have a particular disease.

### TECHNICAL FIELD

The invention relates to prediction of neurological treatment by utilizing machine learning, voice recognition, and MRI derived brain biomarkers.

### BACKGROUND

Neurodegeneration is a term used to describe a wide range of conditions and diseases which primarily affect the neurons in the human brain. Strictly speaking, neurodegeneration refers to the progressive atrophy (death) and loss of function of neurons, which is present in neurodegenerative diseases such as Alzheimer's disease, Huntington's disease, and Parkinson's disease. It is important to note that neurons normally do not regenerate, replace, or reproduce themselves, so when they become damaged or die they cannot be replaced by the body. Neurodegenerative diseases are incurable and debilitating conditions that result in progressive degeneration and/or death of nerve cells. This causes problems with movement (called ataxias), or mental functioning (called dementias). Frequently, both movement and mental symptoms are present in patients suffering from neurodegenerative disorders. Dementias are responsible for the greatest burden of neurodegenerative diseases, with Alzheimer's representing approximately 60-70% of dementia cases. There is a need to be able to decrease the time to diagnosis as well as diagnose the above mentioned disorders at an earlier stage in order to increase the efficacy of treatment and improve clinical outcomes.

### SUMMARY OF THE INVENTION

There are known several attempts to develop computer systems to provide treatment recommendations.

Current problems associated with treatment of neurodegenerative conditions are a result of limited diagnostic information about a given patient. Physicians rely on history, physical exam, blood work, and brain MRI to arrive at a diagnosis and make treatment recommendations. This is a limited approach as the sensitivity of the analysis is restricted by many factors such as; the ability of the physician to discern visible pathology from the imaging and correlate it with a disease process and stage of disease; ability to identify mild subclinical speech anomalies; ability of the physician to quantify and correlate lab work data with a specific disease process (e.g. CBC, CMP, CRP in Huntington's versus Alzheimer's); and finally the physician's ability to take multiple data points from various diagnostic modalities (e.g. genetic testing, CBC, BMP, CRP, MRI, BMI, speech, etc.) and create a health “portrait” of a given patient. Currently, the physician not only does not have the ability to create such an objective health “portrait”, but they also cannot compare it to a database of many such health “portraits” with known neurological diagnosis and just as importantly know the historical outcomes of treatment for those individuals. Key MRI components of the health “portrait” of an individual with neurodegenerative process are brain age, brain health, and the gyrification coefficient. Brain age, defined as the difference between the estimated age and the biological age of the individual, has been suggested to be a reliable, MRI scanner-independent, and efficient measure of deviation from normal (statistically speaking) brain aging in healthy participants and to be able to predict individual brain maturation. Different research groups found brain age to be correlated with physical fitness, mortality risk in elderly participants, and human immunodeficiency virus status, and cognitive performance Others have shown that, compared with control groups, brain age was higher in patients with psychiatric disorders, mild cognitive impairment, Alzheimer' s disease, or diabetes and much lower for long-term meditators. Taken together, these findings provide strong evidence that age can be estimated using features from structural brain imaging and can be meaningfully related to other age-related processes. Brain health is based on an evaluation of the combined effects of whole brain tissue atrophy (brain wasting secondary to nerve cell death) and vascular disease in a single measure. About 15% of the freshly oxygenated blood pumped out by the heart goes to the brain. The brain itself has a very robust network of blood vessels and capillaries. These vessels are prone to disease as a result of high blood pressure, aging, high cholesterol, diabetes and other disorders. Presence of small vessel disease and brain tissue atrophy (death) both increase with age, are often present together, and are risk factors for stroke, dementia, and neurodegeneration. The importance of vascular disease on accelerating neurodegenerative pathologies and cognitive decline has recently been recognized. Moreover, structural changes in the brain are rarely (if at all) monitored in a continuous way.

Using longitudinal data enables tracking of long-term changes in the patient's brain and makes diagnosis more accurate.

In one aspect, the invention relates to a computer-implemented method for predicting neurological treatment for a patient, the method comprising: analyzing a pre-stored brain image of the patient by means of a Convolutional Neural Network to determine brain image analysis result including at least one of: a presence of a tumor or lesion, brain age, brain health, gyrification coefficient; receiving additional data, including at least one of: voice recognition index, additional symptom checks, blood work results, genetic sequencing results; and combining the brain image analysis result with the additional data to determine a score related to a probability that the patient may have a particular disease.

In another aspect, the invention relates to a computer-implemented method for determining a recommended neurological treatment for an examined patient, the method comprising: receiving examined patent's health data comprising a three-dimensional brain image of the examined patient and additional data comprising at least one of: voice recognition index, additional symptom checks, blood work results and genetic sequencing results; processing the three-dimensional brain image of the examined patient by a pre-trained Convolutional Neural Network to determine a brain image analysis result including at least one of: a presence of a tumor or lesion, brain age and brain health; comparing the brain image analysis result and the additional data of the examined patient with other patients' health data read from other patents' database to determine at least one other patient as a closest matching patient having at least some types of the health data close to the health data of the examined patient; and determining at least one treatment of the at least one closest matching patient and presenting that at least one treatment as the recommended neurological treatment for the examined patient.

There is also disclosed a computer-implemented system for performing the method described herein.

These and other features, aspects and advantages of the invention will become better understood with reference to the following drawings, descriptions and claims.

### BRIEF DESCRIPTION OF DRAWINGS

Various embodiments are herein described, by way of example only, with reference to the accompanying drawings, wherein:

FIG. 1 shows an overview of the system in accordance with one embodiment;

FIG. 2 shows a brain age determining convolutional neural network (CNN) architecture in accordance with one embodiment;

FIG. 3 shows a neural network training procedure;

FIG. 4 shows a neural network inference procedure;

FIG. 5 shows a method of operation of the scoring module and the treatment recommendation module; and

FIG. 6 shows a computer-implemented system in accordance with one embodiment.

### DETAILED DESCRIPTION OF THE EMBODIMENTS

The computer system and method as presented herein is intended, in accordance with certain embodiments, to augment the work of a physician and is not intended to replace the doctor. The system, and in particular its scoring module 130, in certain embodiments, analyzes multiple data points derived from diagnostic tests and creates a health “portrait” of an individual suspected of having a neurodegenerative condition. This “portrait” is then compared to a large data set composed of normal and pathological “portraits” of individuals with neurodegenerative conditions. With a large amount of cases for unremarkable (healthy) brains and brains with symptoms of a range of conditions, the system is able to place the patient on a spectrum for a wide range of indicators signaling the presence and in some cases also the severity of the condition of the patient. The goal of the computer system is to provide an objective datapoint that augments the physician's decision making and diagnostic capabilities, which are based on traditional approaches of medical practice (e.g. history and physical).

Therefore, the system can, in certain embodiments, provide:
objective, automated analysis with specific, interpretable measurements comparable across patients;
capability to perform the comparative analysis with a vast database of scans of both healthy and problematic cases in separate age groups;
the capability to perform a trend analysis—growing/shrinking/changes of structures, tumors, pathologies etc.;
time savings, as the process is automated;
multimodal analysis (multiple sequences at the same time, registered) in the case of some functionalities (brain health), imaging+voice/speech analysis.

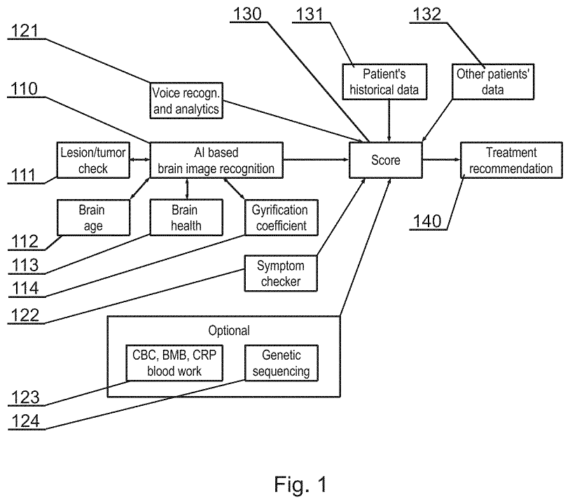

The system of the invention is shown in an overview on FIG. 1. It comprises the following modules in certain embodiments.

An AI-based (artificial intelligence) brain image recognition module 110 is configured to process brain image data to determine various parameters related to the brain. It may comprise at least one of the following sub-modules. A lesion/tumor check sub-module 111 is configured to check whether the brain image is indicative of any tumors or lesions, and if so, what is their number, location, shape and volume. A brain age sub-module 112 is configured to compute a brain age, and in addition it may provide information on which areas of the brain contributed to the decision related to the particular brain age (therefore, it may indicate where the potential cause of problem is located). A brain health sub-module 113 is configured to determine potentially healthy and unhealthy brain areas, such as by quantifying visible brain injury from small vessel disease and brain atrophy. A gyrification coefficient module 114 is configured to determine a gyrification index that indicates the amount of folding of the surface of the brain, which can be provided as an overall (global) gyrification index and/or a local gyrification index (cortical folding) (relating to particular areas of the brain surface) (the role of gyrification index is described e.g. in a publication by Jonathan M Harris et al. “Abnormal cortical folding in high-risk individuals: a predictor of the development of schizophrenia?” (Biological Psychiatry, Volume 56, Issue 3, 1 Aug. 2004, Pages 182-189)).

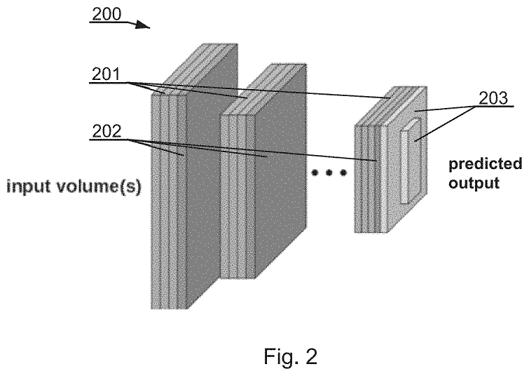

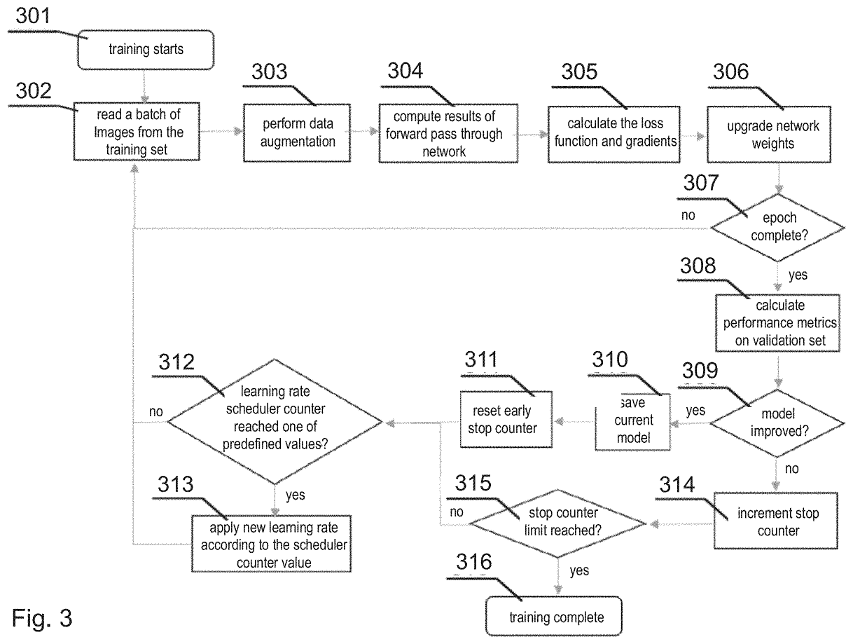

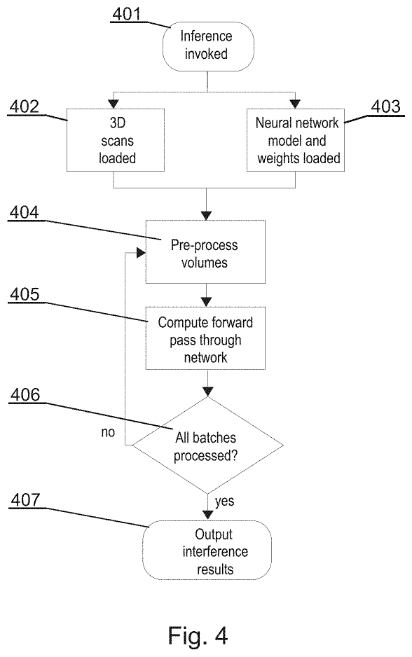

The brain image recognition module 110 and its sub-modules 111-113 may be implemented by means of at least one Convolutional Neural Network (CNN) 200, such as shown in FIG. 2, while the sub-module 114 may be implemented as an algorithmic module. The network training process is presented with reference to FIG. 3 and the network inference process is presented with reference to FIG. 4. The network shown has a contracting path comprising 3D convolutional layers 201, pooling layers 202 and dense layers 203. Each convolutional layer 202 has a plurality of filters (for example, from 16 to 512 filters). Convolutions may be of the regular kind or dilated convolutions. A different stride of S_{C} (for example: 1, 2, 4 or 8) can be set for each convolutional layer 201. The 3D maximum pooling layers 202 may have optional added dropout or other regularization. A different stride of S_{MP} (for example, 1, 2, 4 or 8) can be set for each pooling layer 202. The dense layers 203 may have an optionally added dropout or other regularization.

The network is configured to accept as a primary input one or more medical images (preferably, 3D volumes) of the brain to be analyzed. For example, different image types of the brain can be provided, such as T1 and T2-weighted volumes, or images made with and without contrast. Once the network is trained for the specific task, it can provide, as output, a parameter of the particular sub-module 111-113 as discussed above.

The training process of the CNN 200 used in certain embodiments for the brain image recognition module 110 is carried out as shown in FIG. 3. The objective of the training for the CNN 200 is to tune the parameters of the CNN 200, so that the network is able to determine the parameters corresponding to the modules 111-113.

In a specific embodiment, the brain image recognition module 110 may comprise a single CNN configured to determine all the requested brain parameters of modules 111-113.

In another specific embodiment, the brain image recognition module 110 may comprise a plurality of CNNs, each configured to determine a distinct type of brain parameters, namely each configured to function as a separate module 111-113.

The following description will provide an overall method for training of the CNN, regardless of whether it is configured to output all parameters or one type of parameters.

The training database may be split into a training set used to train the model, a validation set used to quantify the quality of the model, and a test set. The training database may comprise a plurality of 3D volumes (3D scans of a brain) of:
brains with one or more of lesions and tumors, annotated with descriptors indicating at least one of: location, shape, volume, MR signal intensity, known diagnosis (either confirmed via pathology or expert consensus) in order to train the CNN for determining the type of lesion and tumor (module 111 functionality);thus serving a predictive analytics function
brains with one or more of lesions and tumors, annotated with descriptors indicating at least one of: location, shape, volume, MR signal intensity, diagnosis (either confirmed via pathology or expert consensus) in order to train the CNN for determining the prognosis of lesion and tumor (module 111 or 130 functionality); thus serving a predictive analytics function
brains with one or more of lesions and tumors, annotated with descriptors indicating at least one of: location, shape and volume, MR signal intensity, known diagnosis (either confirmed via pathology or expert consensus), known treatment outcome (either confirmed via actual treatment outcomes or expert consensus) in order to train the CNN for determining the treatment recommendation of lesion and tumor (module 111 or 130 functionality); thus serving a predictive analytics function
healthy individuals annotated with a known biological age, in order to train the CNN for determining the brain age (module 112 functionality);
healthy individuals annotated over time with a known medical history, in order to train the CNN for determining normal brain aging (module 112 functionality);
unhealthy individuals annotated with a known biological age, in order to train the CNN for determining pathological brain aging (module 112 functionality);
brains with injured areas (such as reactive zones, atrophy, scar, plaques, micro vascular disease, stroke, or trauma), annotated with descriptors indicating the injury type and location, in order to train the CNN for brain health recognition (module 113 functionality);

The training starts at 301. At 302, training volumes are read from the training set, for example one volume at a time.

At 303 the original scans can be augmented. Data augmentation is performed on these scans to make the training set more diverse. The input and output pair of three dimensional volumes is subjected to the same combination of transformations.

At 304, the original 3D volumes and the augmented 3D volumes are then passed through the layers of the CNN in a standard forward pass. The forward pass returns the results, which are then used to calculate at 305 the value of the loss function (i.e., the difference between the desired output and the output computed by the CNN). The difference can be expressed using a similarity metric (e.g., mean squared error, mean average error, categorical cross-entropy, or another metric).

At 306, weights are updated as per the specified optimizer and optimizer learning rate. The loss may be calculated using a per-pixel cross-entropy loss function and the Adam update rule.

The loss is also back-propagated through the network, and the gradients are computed. Based on the gradient values, the network weights are updated. The process, beginning with the 3D volumes batch read, is repeated continuously until an end of the training session is reached at 307.

Then, at 308, in accordance with certain embodiments, the performance metrics are calculated using a validation dataset—which is not explicitly used in training. This is done in order to check at 309 whether not the model has improved. If it is not the case, the early stop counter is incremented by one at 314, as long as its value has not reached a predefined maximum number of epochs at 315. The training process continues until there is no further improvement obtained at 316. Then the model is saved at 310 for further use, and the early stop counter is reset at 311. As the final step in a session, learning rate scheduling can be applied. The session at which the rate is to be changed are predefined. Once one of the session numbers is reached at 312, the learning rate is set to one associated with this specific session number at 313.

Once the training process is complete, the network can be used for inference (i.e., utilizing a trained model for determining a particular parameter). The model can be saved and reused, training doesn't need to be performed before each use.

In other words, the neural network adjusts its internal parameters, which include the weights in the internal convolutional layers of the dimensions W×H×D (they include 3D convolutional kernels), which denote the width and height and depth, respectively, with W, H and D being positive integers and the weights of the additional fully connected layers. During training, the network repeatedly performs the following steps in certain embodiments:
the step of determining the parameter based in the input imaging data,
the computation of the difference between the actual input parameter (as annotated in the training data) and the determined output parameter
The update of weights according to the gradient back-propagation method based on the steepest descent gradient algorithm or one of its variants (Adam, Nadam, adagrad, . . . )

Doing so, the network adjusts its parameters and improves its predictions over time. During training, the following means of improving the training accuracy can be used in certain embodiments:
Learning rate scheduling
Early stopping
Regularization by dropout
L2 regularization
Data augmentation (by random volume rotations, intensity changes, noise introduction, affine transformations etc.)

The training process includes periodic check of the output parameter determination accuracy using a held out input data set (the validation set) not included in the training data. If the check reveals that the accuracy on the validation set is better than the one achieved during the previous check, the complete neural network weights are stored for further use. The early stopping function may terminate the training if there is no improvement observed during the last checks. Otherwise, the training is terminated after a predefined number of steps.

The approach presented herein has the advantage that it does not require significant pre-processing of input data. This makes the system less complex.

The input data is normalized from the raw input data to a range from 0 . . . 1. Preferably, before the normalization, the extreme top and bottom values may be clipped (discarded), to get rid of outlying values.

FIG. 4 shows a flowchart of an inference process for the CNN 200.

After inference is invoked at 401, an input 3D image is loaded at 402 and the CNN 200 and its weights are loaded at 403.

At 404, the input volume is preprocessed (e.g., normalized, cropped, etc.) using the same parameters that were utilized during training.

At 405, a forward pass through the CNN 200 is computed.

At 406, if not all batches have been processed, a new batch is added to the processing pipeline until inference has been performed at all input volumes.

Finally, at 407, the inference results are saved and the output is provided as one or more determined parameters according to the functionality of modules 111-113.

In certain embodiments, the system further comprises the following additional modules.

A voice recognition and analytics module 121 may analyze the patient's capability to retell a story that the patient heard/seen on audio/video or read as a text. This can be a strong indicator of Alzheimer's. The module can operate as explained e.g. in a publication by Juciclara Rinaldi et al, “Textual reading comprehension and naming in Alzheimer's disease patients” (Dement Neuropsychol. 2008 April-June; 2(2): 131-138). The output can be provided e.g. as a value indicating the capabilities of the patient, e.g. on a scale from 1 to 10.

A symptom checker module 122 is configured to determine additional symptoms such as blurry vision, speech disorders, hypertension, memory problems, headaches. This can be done by simply asking the patient a series of simple yes/no questions related to particular symptoms. The output data is used as additional input to the machine learning algorithms and makes diagnoses and predictions more accurate.

The blood work module 123 may be used to input to the system values of results of blood tests, such as CBC, BMP, CRP.

The genetic sequencing module 124 may be used to input to the system DNA data, which may be indicative of heritable neurological disorders.

The scoring module 130 operates as described in the initial section of this detailed description, to determine a score related to a probability that the patient may have a particular disease, based on a health “portrait” of the patient, comprising the inputs from the module 110 (in particular, the parameters output by specific sub-modules 111-114), and at least one of the other modules 121-124. As a result, using more input signals for prediction may result in increased accuracy.

The scoring module 130 is connected to other patients' database 132 that contains health “portraits” of other patients, comprising one or more data corresponding to the data provided by the modules 110, 121-124.

The system may make a decision based not only on the current data, but also on historical data of the examined patient stored in database 131 and of the other patients stored in database 132. Therefore, combining multi-domain data to make a final diagnosis and tracking long-time changes of crucial coefficients and marker levels is very beneficial.

Finally, a treatment recommendation module 140 recommends predicted treatment.

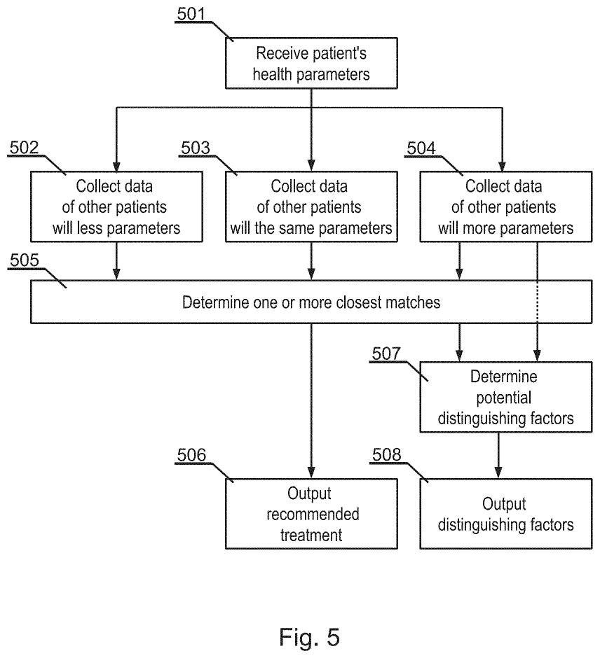

In a specific embodiment, the scoring module 130 and the treatment recommendation module 140 may operate according to the procedure shown in FIG. 5. The scoring module 130 receives in step 501 the health “portrait” of the patient, namely a set of parameters from the module 110 and at least one of the other modules 121-124. Next, the scoring module sends enquiries to the other patients' database 132 to collect data of other patients:
for whom the same set of health parameters is available (step 503) and whose parameters are close to the examined patient's parameters;
for whom a smaller set of health parameters is available (step 502) and whose parameters are close to the examined patient's parameters;
for whom a larger set of health parameters is available (step 504) and whose parameters are close to the examined patient's parameters.

For an illustrative example, consider a case when the examine patent's health “portrait” comprises data on a lesion location and parameters (generated by module 111), brain age (generated by module 112) and blood work results (generated by module 123).

In step 503 the procedure selects, from the other patients' database 132, data related to the patients for whom the same set of parameters are available, namely only the lesion location and parameters, brain age and blood work results and wherein these parameters are close to the examined patent's parameters. The measure of how close the parameters are may be determined by a threshold value unique for each parameter, for example:
for a lesion, the threshold parameter may require the other patient's lesion to be within the brain region (or, for a more closer match, within the same blood vessel) and to be of a similar size (e.g. +/−20% of the narrowing measure)
for a tumor, the threshold parameter may require the other patient's tumor to be within the brain region (or, for a more closer match, within the same blood vessel) and to be of a similar size (e.g. +/−20% of the tumor volume)
for a brain age, the threshold parameter may require the other patient's brain age to be within +/−3 years;
for a gyrification index, the threshold parameter may require the other patient's gyrification index to be within +/−10% of gyrification value;
for a symptom check, the threshold parameter may require the other patient to have the same symptom (or, if the symptom has a measure parameter, to have the parameter within a +/−10% range);
for voice recognition and analytics results, the threshold parameter may require the other patient to have the same symptom (such as slower speech);
for blood work results, the threshold parameter may require the other patient to have particular blood work results within a given percentage range (individual for each result);
for genetic sequencing data, the threshold parameter may require the other patient to have a particular gene fragment.

In the present example, as an output of step 503, the data of other patients who have close lesion examination results, close brain age and close blood results will be returned.

In step 502, the data of patients who have less parameters available will be returned. For step 502, a list of essential parameters (such as lesion location and parameters, brain age) and optional parameters (such as blood work results) may be defined. Therefore, as an output of step 502, the data of other patients who have close lesion examination results and close brain age, but no blood results will be returned.

In step 504, the data of patients who have more parameters available will be returned. For step 504, a list of essential parameters (such as lesion location and parameters, brain age) may be defined, in that case the patients who have at lease the essential parameters and optional parameters will be analyzed. Therefore, as an output of step 504, the data of other patients who have close lesion examination results and close brain age will be returned along with their other health parameters.

In step 505, a closest matching other patient is determined among the other patients data output from steps 502, 503, 504. The closest matching patient can be a patent having the same health “portrait” as the examined patient. However, in case there are many other patients having a close (but not as exact) health portrait, the other health portraits may be selected based on probability. Therefore, the closest match can be either a patient of the closest set of parameters, or a patient (or a group of patients) from a largest group that has the closest parameters to that of the examined patients. Step 505 can be realized either as a matching algorithm or as a neural network trained to find the closest match (for example, a neural network having a structure of FIG. 2 and trained according to FIG. 3 and configured to perform inference according to FIG. 4). Step 505 may output one or more of the closest matches (i.e. records of other patients data) as different options along with the definition of selection criteria (e.g. closest match, closest group).

In step 506, the treatment recommendation module 140 outputs a recommended treatment, based on the data of the closest matches obtained in step 505. For example, the recommended treatment may be the same treatment as indicated as successful in the records of other patients data for the closest match. However, in case the closest matches include a plurality of records, the treatment recommendation module may analyze the treatments indicated as successful and unsuccessful for these patients and output, as a recommendation, a list of treatments sorted according to a probability of success.

Optionally, the closest matches may be further analyzed in step 507 in combination with data of patients whose records indicated more health parameters than that for the examined patient. This may be important especially in case of patients for whom there are not many parameters available. In step 507, the data of other patients with similar symptoms may be analyzed to determine what other parameters are most common that would allow to provide a more detailed diagnosis. Correspondingly, step 508 may output a list of distinguishing factors, i.e. health parameters which when examined would allow to determine a closer match than for the current record of the patient.

Alternatively, if a patient lacks significant data from their health portrait, (ex. Only MRI and basic demographic data are available) the CNN will attempt to predict the possible result(s) of the missing parameters.

Alternatively, if a patient lacks significant data from their health portrait, (ex. Only MRI and basic demographic data are available) the CNN will attempt to suggest the basic minimum inputs necessary to increase diagnostic yield.

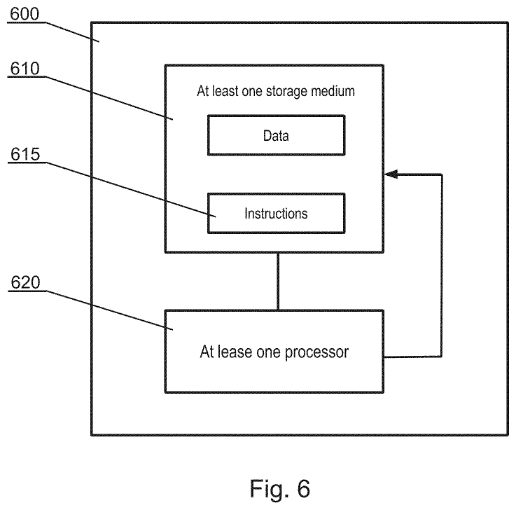

The functionality of the system of FIG. 1 can be implemented in a computer-implemented system 300, such as shown in FIG. 6. The system may include at least one non-transitory processor-readable storage medium that stores at least one of processor-executable instructions or data and at least one processor communicably coupled to the at least one non-transitory processor-readable storage medium. At least one processor is configured to perform the steps of the methods presented herein.

The computer-implemented system 600, for example a machine-learning system, may include at least one non-transitory processor-readable storage medium 610 that stores at least one of processor-executable instructions 615 or data; and at least one processor 620 communicably coupled to the at least one non-transitory processor-readable storage medium 610. The at least one processor 620 may be configured (by executing the instructions 615) to perform the functionality of the modules of FIG. 1 and the procedure of FIG. 5.

Although the invention is presented in the drawings and the description and in relation to its preferred embodiments, these embodiments do not restrict nor limit the invention. It is therefore evident that changes, which come within the meaning and range of equivalency of the essence of the invention, may be made. The presented embodiments are therefore to be considered in all aspects as illustrative and not restrictive. According to the abovementioned, the scope of the invention is not restricted to the presented embodiments but is indicated by the appended claims.

What is claimed is:

1. A computer-implemented method for determining a recommended neurological treatment for an examined patient, the method comprising:
receiving examined patent's health data comprising a three-dimensional brain image of the examined patient and additional data comprising at least one of: voice recognition index, additional symptom checks, blood work results and genetic sequencing results;
processing the three-dimensional brain image of the examined patient by a pre-trained Convolutional Neural Network to determine a brain image analysis result including at least one of: a presence of a tumor or lesion, brain age and brain health;
comparing the brain image analysis result and the additional data of the examined patient with other patients' health data read from other patents' database to determine at least one other patient as a closest matching patient having at least some types of the health data close to the health data of the examined patient; and
determining at least one treatment of the at least one closest matching patient and presenting that at least one treatment as the recommended neurological treatment for the examined patient.

2. The method according to claim 1, further comprising processing the three-dimensional brain image of the examined patient by an algorithmic module to determine a gyrification coefficient as a component of the brain image analysis result.

3. The method according to claim 1, further comprising pre-training the Convolutional Neural Network used to determine the brain image analysis result by data of a training database, comprising at least one of the following data sets of three-dimensional brain images of:
brains with one or more of lesions and tumors, annotated with descriptors indicating at least one of: location, shape, volume, magnetic resonance signal intensity and known diagnosis for determining a presence of the tumor or lesion;
brains of healthy individuals annotated with a known biological age for determining the brain age; and
brains with injured areas annotated with descriptors indicating the injury type and location, for determining brain health.

4. The method according to claim 3, comprising using a distinct convolutional neural network for determining each of the presence of the tumor or lesion, the brain age and the brain health.

5. The method according to claim 1, further comprising comparing the brain image analysis result and the additional data of the examined patient with other patients' health data read from other patents' database to determine at least one other patient as a closest matching patient by means of a convolutional neural network pre-trained by at least one of the following data sets of three-dimensional brain images of: brains with one or more of lesions and tumors, annotated with descriptors indicating at least one of: location, shape, volume, MR signal intensity, diagnosis for determining the treatment recommendation of lesion and tumor.

6. The method according to claim 1, further comprising comparing the presently examined and historical data of the examined patient read from patient's historical database with other patients' health data including other patients' historical data read from the other patents' database.

7. The method according to claim 1, comprising determining the at least one other patient as the closest matching patient having the same types of the health data close to all types of the health data of the examined patient.

8. The method according to claim 1, comprising determining the at least one other patient as the closest matching patient having less types of health data close to the health data of the examined patient.

9. The method according to claim 1, comprising determining the at least one other patient as the closest matching patient having the same types of the health data close to all types of the health data of the examined patient and additional types of the health data.

10. The method according to claim 9, further comprising, determining the additional types of the health data known for the at least one other patient determined as the closest matching patent and presenting the health data as distinguishing factors of the at least one closest matching patient.

11. The method according to claim 1, comprising determining the health data as close when the health data are not different than a predefined threshold value.

12. A computer-implemented system comprising:
at least one nontransitory processor-readable storage medium that stores at least one of processor-executable instructions or data; and
at least one processor communicably coupled to at least one nontransitory processor-readable storage medium, wherein at least one processor is configured to perform the steps of the method of claim 1.

13. A computer-implemented method for predicting neurological treatment for a patient, the method comprising:
analyzing a pre-stored brain image of the patient by means of a Convolutional Neural Network (CNN) to determine a brain image analysis result including at least two of: a presence of a tumor or lesion, brain age, brain health, a gyrification coefficient;
receiving additional data, including at least one of: voice recognition index, additional symptom checks, blood work results, genetic sequencing results; and
combining the brain image analysis result with the additional data to determine a value of probability that the patient may have a particular disease.

14. A computer-implemented system comprising:
at least one nontransitory processor-readable storage medium that stores at least one of processor-executable instructions or data; and
at least one processor communicably coupled to at least one nontransitory processor-readable storage medium, wherein at least one processor is configured to perform the steps of the method of claim 13.
